(19) United States
(12) Patent Application Publication
(10) Pub. No.: US 2004/0063696 A1
(43) Pub. Date: Apr. 1, 2004

(54) 1,1-dioxo-2h-1,2-benzothiazine-3-carboxamide derivatives,method for preparing same and pharmaceutical compositions comprising same

(76) Inventors: Jean-Claude Madelmont, Romagnat (FR); Isabelle Giraud, Dourdan (FR); Aurelien Vidal, Ceyrat (FR); Emmanuelle Mounetou, Clermont-Ferrand (FR); Maryse Rapp, Veyre-Monton (FR); Jean-Claude Maurizis, Perignat-Les-Sarlieve (FR); Pierre Renard, Le Chesnay (FR); Daniel-Henri Caignard, Le Pecq (FR); Jean-Guy Bizot-Espiard, Paris (FR)

Correspondence Address: THE FIRM OF HUESCHEN AND SAGE, 500 COLUMBIA PLAZA, 350 EAST MICHIGAN AVENUE, KALAMAZOO, MI, 49007, US

(21) Appl. No.: 10/451,489

(22) PCT Filed: Dec. 21, 2001
(86) PCT No.: PCT/FR01/04135

(30) Foreign Application Priority Data
Dec. 21, 2000 (FR) 00/16739

(51) Int. Cl. A61K 31/5415; C07D 279/16

(52) U.S. Cl. 514/226.5; 544/49

(57) ABSTRACT

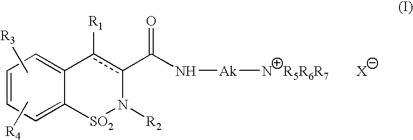

wherein: [symbol] [symbol] represents a single or double bond, [symbol] R₁ represents a hydrogen atom or a hydroxy, alkoxy, acyloxy, alkylsulphonyloxy, arylsulphonyloxy or arylalkoxy group, [symbol] R₂ represents a hydrogen atom or an alkyl group, [symbol] R₃ and R4, which may be identical or different, each represent a hydrogen atom, a halogen atom or an alkyl, hydroxy or alkoxy group, [symbol] Ak represents an alkylene chain, [symbol] R₅, R6 and R₇, which may be identical or different, each represent an alkyl group, or R₅, R6 and R₇, taken together with the nitrogen atom carrying them, form a saturated or unsaturated nitrogen-containing heterocycle,

[symbol]

X represents a halogen atom, and its optical isomers when they exist. Medicaments.

The present invention relates to new 1,1-dioxo-2H-1,2-benzothiazine-3-carboxamide compounds, to a process for their preparation and to pharmaceutical compositions containing them, and to their use in the treatment of pathologies of the cartilage.

The anti-inflammatories currently commercially available for the treatment of articular pathologies, such as arthritis or arthrosis, generally exhibit a low affinity for the target tissues and require the administration of high doses to achieve the desired therapeutic effect.

The administration of such strong doses of active ingredients gives rise to an increase in the frequency of side effects. For example, the administration of non-steroidal anti-inflammatories is known to cause significant gastrointestinal toxicity.

There has thus been particular interest in obtaining new compounds that are capable of specifically targeting cartilaginous tissue and thus limiting, or even suppressing, the undesirable effects observed with existing anti-inflammatories.

1,1-Dioxo-2H-1,2-benzothiazine-3-carboxamide compounds and their potential use in the treatment of pathologies of the cartilage have been described in J. Med. Chem. 1999, 42 5235-40.

However, those compounds have only moderate activity with respect to the expression of cartilage-protecting proteoglycans.

The new compounds forming the subject of the present invention retain the very strong affinity for cartilaginous tissues already described for the compounds of the prior art but, in addition, they possess cartilage-protecting properties that are very clearly superior to those of the compounds already described, which, in view of the similarity of their structures, could not have been foreseen at all. These properties therefore make the compounds of the invention extremely useful in the treatment of pathologies such as arthritis or arthrosis.

More specifically, the present invention relates to compounds of formula (I):

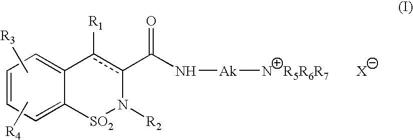

wherein:

represents a single or double bond,

R₁ represents a hydrogen atom or a hydroxy group, a linear or branched (C₁-C₆)alkoxy group, a linear or branched (C₁-C₆)acyloxy group, a linear or branched (C₁-C₆)alkylsulphonyloxy group, an arylsulphonyloxy group or an aryl-(C₁-C₆)alkoxy group in which the alkoxy moiety is linear or branched,

R₂ represents a hydrogen atom or a linear or branched (C₁-C₆)alkyl group,

R₃ and R₄, which may be identical or different, each represent a hydrogen atom, a halogen atom or a linear or branched (C₁-C₆)alkyl group, a hydroxy group or a linear or branched (C₁-C₆)alkoxy group,

Ak represents a linear or branched (C₁-C₆)alkylene chain,

R₅, R₆ and R₇, which may be identical or different, each represent a linear or branched (C₁-C₆)alkyl group,

or R₅, R₆ and R₇, taken together with the nitrogen atom carrying them, form a saturated or unsaturated nitrogen-containing heterocycle,

X represents a halogen atom, and its optical isomers when they exist, excluding compounds wherein, simultaneously, [symbol] represents a double bond, R₁ represents a hydroxy group, R₂, R₅ and R₆ each represent a methyl group, R₃ and R₄ each represent a hydrogen atom, and Ak represents a —(CH₂)₃— group.

“Saturated or unsaturated nitrogen-containing heterocycle” is to be understood as meaning a saturated or unsaturated, aromatic or non-aromatic, monocyclic group having from 5 to 7 ring members, containing one, two or three hetero atoms, one of those hetero atoms being a nitrogen atom, and the additional hetero atom(s) that is/are optionally present being selected from the atoms oxygen, nitrogen and sulphur, it being understood that the nitrogen-containing heterocycle can optionally be substituted by one or more identical or different linear or branched (C₁-C₆)alkyl groups. The preferred nitrogen-containing heterocycles are the groups pyridyl and piperidyl that is N-substituted by a linear or branched (C₁-C₆)alkyl group.

The preferred compounds of formula (I) are those wherein R₂ represents a linear or branched (C₁-C₆)alkyl group.

The preferred compounds of formula (I) are those wherein X represents an iodine atom.

According to an advantageous embodiment, the preferred compounds of the invention are those wherein R₆ and R₇, which may be identical or different, each represent a linear or branched (C₂-C₆)alkyl group.

Among the preferred compounds of the invention, there may be mentioned {3-[(4hydroxy-2-methyl-1,1 -dioxo-2H- 1,2-benzothiazin-3-yl)-carbonylamino]-propyl}diethylmethylammonium iodide.

The invention relates also to a process for the preparation of compounds of formula (I), characterised in that a compound of formula (II):

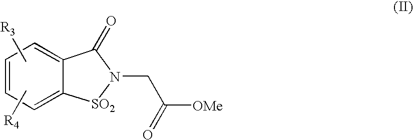

wherein R₃ and R₄ are as defined for formula (I), is reacted in the presence of a base, to yield a compound of formula (III):

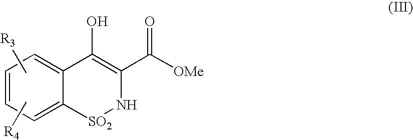

wherein R₃ and R₄ are as defined hereinbefore,

which is reacted, if desired, with a compound of formula (IV):

R′₂—Y₁ (IV)

wherein R′₂ represents a linear or branched (C₁-C₆)alkyl group, and Y₁ represents a leaving group conventional in organic chemistry, to yield a compound of formula (V):

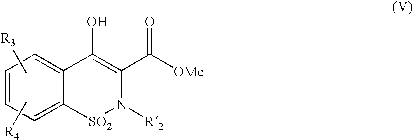

wherein R′₂, R₃ and R₄ are as defined hereinbefore,

which compounds of formula (III) or (V) are reacted, if desired, with a compound of formula (VI):

R′₁—Y₂ (VI)

wherein R′₁ represents a linear or branched (C₁-C₆)alkyl group, a linear or branched (C₁-C₆)acyl group, a linear or branched (C₁-C₆)alkylsulphonyl group, an arylsulphonyl group or an aryl-(C₁-C₆)alkyl group in which the alkyl moiety is linear or branched, and Y₂ represents a leaving group customary in organic chemistry, to yield a compound of formula (VII):

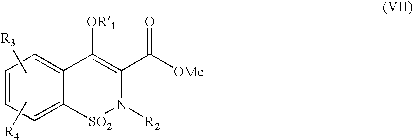

wherein R′₁, R₂, R₃ and R₄ are as defined hereinbefore,

or with an appropriate reducing agent, to yield a compound of formula (VIII):

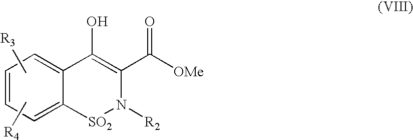

wherein R₂, R₃ and R₄ are as defined hereinbefore,

which is optionally converted:

by elimination, to a compound of formula (IX), under conventional conditions of organic chemistry

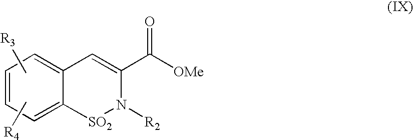

wherein R₂, R₃ and R₄ are as defined hereinbefore,

which is reduced, if desired, to a compound of formula (X):

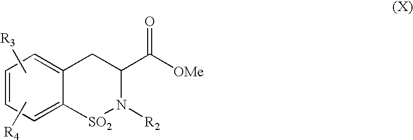

wherein R₂, R₃ and R₄ are as defined hereinbefore,

or by reaction with a compound of formula (VI), to yield a compound of formula (XI):

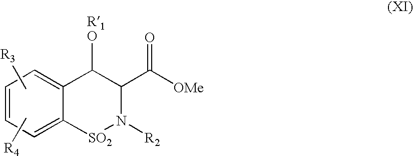

wherein R′₁, R₂, R₃ and R₄ are as defined hereinbefore,

which compounds of formulae (III), (V), (VII), (VIII), (IX), (X) and (XI) constitute the totality of the compounds of formula (XII):

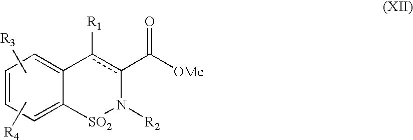

wherein, R₁, R₂, R₃ and R₄ are as defined for formula (I), which are converted to a compound of formula (XIII):

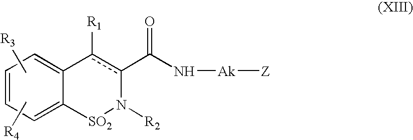

wherein R₁, R₂, R₃ and R₄ are as defined hereinbefore, Ak is as defined for formula (I), and Z represents either a group X as defined for formula (I), or a group NR′₅R′₆ wherein R′₅ and R′₆, which may be identical or different, each represent a linear or branched (C₁-C₆)alkyl group or together form a saturated or unsaturated, non-aromatic nitrogen-containing heterocycle,

which is reacted:

when Z represents a group NR′₅R′₆ as defined hereinbefore, with a compound of formula (XIV):

R′₇—X (XIV)

wherein R′₇ represents a linear or branched (C₁-C₆)alkyl group, and X is as defined for formula (I),

to yield a compound of formula (Ia), a particular case of the compounds of formula (I):

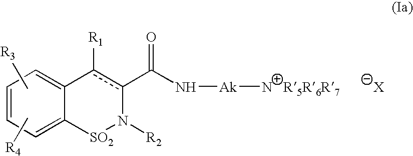

wherein R₁, R₂, R₃, R₄, Ak, R′₅, R′₆, R′₇ and X are as defined hereinbefore,

or, when Z represents a group X as defined hereinbefore, with a compound of formula (XV):

NR″₅R″₆R″₇ (XV)

wherein R″₅, R″₆ and R″₇ form together with the nitrogen atom carrying them an aromatic nitrogen-containing heterocycle,

to yield a compound of formula (Ib), a particular case of the compounds of formula (I):

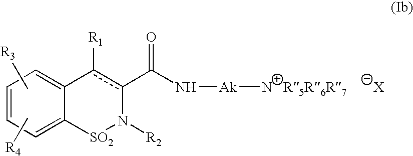

wherein R₁, R₂, R₃, R₄, Ak, R″₅, R″₆, R″₇ and X are as defined hereinbefore,

which compounds of formulae (Ia) and (Ib) constitute the totality of the compounds of formula (I), which are purified, if necessary, in accordance with a conventional purification technique, and which are separated, where appropriate, into their optical isomers in accordance with a conventional separation technique.

The compound of formula (II) is obtained starting from the compound of formula (XVI):

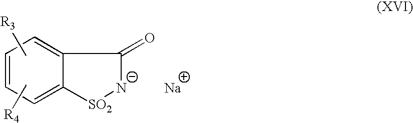

wherein R₃ and R₄ are as defined for formula (I),

in accordance with the process described in J. Med. Chem. 1999, 42, 5235-40. In biological studies, the compounds of the present invention have demonstrated an increased tropism for cartilaginous tissues. Those molecules also have properties in respect of the cartilage which makes them especially useful in the treatment of pathologies such as arthrosis and arthritis.

The invention relates also to pharmaceutical compositions comprising as active ingredient at least one compound of formula (I) with one or more inert, non-toxic, pharmaceutically acceptable carriers. Amongst the pharmaceutical compositions according to the invention there may be mentioned more especially those which are suitable for oral, parenteral (intravenous or sub-cutaneous) or nasal administration, tablets or dragées, sublingual tablets, gelatin capsules, lozenges, suppositories, creams, ointments, dermal gels, injectable preparations, drinkable suspensions, etc..

The useful dosage can be adapted in accordance with the nature and the severity of the disorder, the route of administration and the age and weight of the patient. The dosage ranges from 0.5 mg to 2 g per 24 hours in one or more administrations.

The following Examples illustrate the invention but do not limit it in any way.

The starting materials used are known products or are prepared according to known procedures.

The structures of the compounds described in the Examples were determined in accordance with the customary spectroscopic techniques (infrared, NMR, mass spectrometry (MS)).

### EXAMPLE 1

### {3-[(4-Hydroxy-2-methyl-1,1-dioxo-2H-1,2-benzothiazin-3-yl)carbonylamino]diethylmethylammonium iodide

Step A:

### N-[3-(Diethylamino)-propyl]4-hydroxy-2-methyl-1,1-dioxo-2H-1,2benzothiazine-3-carboxamide

A suspension of 10 mmol of methyl 4-hydroxy-2-methyl-1,1-dioxo-2H-1,2-benzothiazine-3-carboxylate (the preparation of which is described in J. Med. Chem. 1999, 42, 5235-40) and 11 mmol of 3-(diethylamino)propylamine in xylene is refluxed under an inert atmosphere for 18 hours. After cooling and evaporation, the resulting residue is purified by chromatography over silica (eluant: gradient of ethanol in dichloromethane from 0 to 50%, then dichloromethane/ethanol/ammonia 50/48/2), and then recrystallised to yield the expected product in the form of a beige solid.

Melting point: 107-110° C.

Step B:

### {3-[(4-Hydroxy-2-methyl-1,1-dioxo-2H-1,2-benzothiazin-3-yl)-carbonylamino]propyl}diethylmethylammonium iodide

2.5 ml of methyl iodide are added to 10 mmol of the compound described in the preceding Step A suspended in acetone, and then the reaction mixture is refluxed under an inert atmosphere for 6 hours. After returning to room temperature and evaporation of the mixture, the resulting residue is filtered, washed, dried and then recrystallised to yield the expected product in the form of a beige solid.

Melting point: 169-171° C.

### EXAMPLE 2

### {3-[(4-Hydroxy-2-methyl-1,1-dioxo-2H-1,2-benzothiazin-3-yl)carbonylamino]propyl}trimethylammonium iodide

The expected product is obtained according to the process described in Step B of Example 1, starting from the compound described in Step A of Example 1 and ethyl iodide.

Melting point: 204-206° C.

### EXAMPLE 3

### {4-[(4-Hydroxy-2-methyl-1,1-dioxo-2H-1,2-benzothiazin-3-yl)carbonylamino]butyl}trimethylammonium iodide

Step A:

### 4-(Dimethylamino)-butyronitrile

A solution of 10 mmol of 4-bromobutyronitrile in acetonitrile is added, at 50° C., to a suspension of 10 mmol of dimethylamine hydrochloride, 15 mmol of potassium carbonate and 1 mmol of potassium iodide in the same solvent. The mixture is then maintained at that temperature for 16 hours. After evaporation, the resulting residue is taken up in 1N hydrochloric acid. The aqueous phase is washed with ether, neutralised with a 1N sodium hydroxide solution and re-extracted several times with ether. The various organic phases are then combined, dried, filtered and evaporated to yield the expected product in the form of a dark-coloured oil.

Step B:

### 4-(Dimethylamino)-butylamine

A solution of the compound obtained in the preceding Step A (17.8 mmol) in ether is added, at 0° C. under an inert atmosphere, to a suspension of lithium aluminium hydride (26.7 mmol) in ether. The mixture is then returned to room temperature and stirred at that temperature for 1 hour. After hydrolysis according to the method of Mihaïlovic (1 g of water, 1 g of 15% sodium hydroxide and 3 g of water), the reaction mixture is filtered over Celite. Evaporation of the filtrate under reduced pressure enables the expected product to be isolated in the form of a light-coloured oil.

Step C:

### N-[4-(Dimethylamino)-butyl]-4-hydroxy-2-methyl-1,1-dioxo-2H-1,2benzothiazine-3-carboxamide

The expected product is obtained according to the process described in Step A of Example 1, starting from methyl 4-hydroxy-2-methyl-l,1-dioxo-2H-1,2-benzothiazine-3carboxylate (the preparation of which is described in J. Med. Chem. 1999, 42, 5235-40) and the compound obtained in the preceding Step B.

MS (electrospray) m/z: 354.13 [M+H^{+]}

Step D:

### [4-[(4-Hydroxy-2-methyl-1,1-dioxo-2H-1,2-benzothiazin-3-yl)-carbonylamino]amino]butyl}trimethylammonium iodide

The expected product is obtained according to the process described in Step B of Example 1 starting from the compound described in the preceding Step C and methyl iodide.

Melting point: 240-242° C.

### EXAMPLE 4

### {3-[(4-Hydroxy-2-methyl-1,1-dioxo-2H-1,2-benzothiazin-3-yl)-carbonylamino]propyl}pyridinium iodide

Step A:

### N-[3-Hydroxy-propyl]4-hydroxy-2-methyl-1,1-dioxo-2H-1,2-benzothiazine-3-carboxamide

The expected product is obtained according to the process described in Step A of Example 1, starting from methyl 4-hydroxy-2-methyl-1,1 -dioxo-2H-1,2-benzothiazine-3-carboxylate (the preparation of which is described in J. Med. Chem. 1999, 42, 5235-40) and 3-amino-1-propanol.

Step B:

### N-[3-Iodo-propyl]4-hydroxy-2-methyl-1,1-dioxo-2H-1,2-benzothiazine-3-carboxamide

To 15 mmol of triphenylphosphine and 15 mmol of imidazole dissolved in a mixture of ether/acetonitrile 75/25 there are added, in portions and at 0° C., 15 mmol of iodine, and then 10 mmol of the compound described in the preceding Step dissolved in ether. The reaction mixture is then stirred at room temperature for 2 hours, and subsequently hydrolysed and extracted with ethyl acetate. The combined organic phases are washed, dried, filtered and then concentrated. The resulting residue is purified by chromatography over silica (eluant:dichloromethane) to yield the expected product.

Step C:

### {3-[(4-Hydroxy-2-methyl-1,1-dioxo-2H-1,2-benzothiazin-3-yl)-carbonylamino]propyl}pyridinium iodide

The expected product is obtained starting from the compound described in the preceding Step in accordance with the process described in Step B of Example 1, replacing methyl iodide by pyridine.

### EXAMPLE 5

### {3-[(4-Methoxy-2-methyl-1,1-diox-2H-1,2-benzothiazin-3-yl)-carbonylamino]propyl}trimethylammonium iodide

Step A:

### Methyl 4-methoxy-2-methyl-1,1-dioxo-2H-1,2-benzothiazine-3-carboxylate

A suspension of 10 mmol of methyl 4-hydroxy-2-methyl-1,1-dioxo-2H-1,2-benzothiazine3-carboxylate (the preparation of which is described in J. Med. Chem. 1999, 42, 5235-40), 20 mmol of potassium carbonate and 15 mmol of dimethyl sulphate in acetone is stirred at room temperature under an inert atmosphere for 24 hours. After filtration, the reaction mixture is treated with 1 ml of concentrated ammonium hydroxide and then evaporated. The resulting residue is taken up in ethyl acetate and washed with water. The organic phase is then dried, filtered and evaporated. The resulting oily residue is subsequently purified by chromatography over silica (eluant:dichloromethane) to yield the expected product in the form of a light-coloured oil that crystallises.

Melting point: 79-80° C.

Step B:

### N-[3-(Dimethylamino)-propyl]4-methoxy-2-methyl-1,1-dioxo-2H-1,2-benzothiazine-3-carboxamide

The expected product is obtained according to the process described in Step A of Example 1 starting from the compound described in the preceding Step A and 3-(dimethylamino)-propylamine.

MS (electrospray) n/z: 354.15 [M+H⁺]

Step C:

### {3-[(4-Methoxy-2-methyl-1,1-dioxo-2H-1,2-benzothiazin-3-yl)-carbonylamino]propyl}trimethylammonium iodide

The expected product is obtained according to the process described in Step B of Example 1 starting from the compound described in the preceding Step B and methyl iodide.

MS (electrospray) m/z: 368.15 [M⁺]

### EXAMPLE 6

### {3-[(4-Acetoxy-2-methyl-1,1-dioxo-2H-1,2-benzothiazin-3yl)-carbonylamino]propyl}trimethylammonium iodide

Step A:

### Methyl 4-acetoxy-2-methyl-1,1-dioxo-2H-1,2-benzothiazine-3-carboxylate

11.5 mmol of pyridine are added to 10 mmol of methyl 4-hydroxy-2-methyl- 1,1-dioxo-2H1,2-benzothiazine-3-carboxylate (the preparation of which is described in J. Med. Chem. 1999, 42, 523540) dissolved in ether, followed, at 0° C., by 11.5 mmol of acetyl chloride. The reaction mixture is then stirred at room temperature for 5 hours, and subsequently hydrolysed and extracted with ethyl acetate. The combined organic phases are washed, dried, filtered and then concentrated. The resulting residue is purified by chromatography over silica (eluant: dichloromethane) to yield the expected product.

Step B:

### {3-[(4-Acetoxy-2-methyl-1,1-dioxo-2H-1,2-benzothiazin-3-yl)-carbonylamino]propyl{trimethylammonium iodide

The expected product is obtained according to the process described in Steps B and C of Example 5, starting from the compound described in the preceding Step.

### EXAMPLE 7

### {b 3-[(4-Hydroxy-2-methyl-1,1-dioxo-3,4-dihydro-2H-1,2-benzothiazin-3-yl)carbonylamino]propyl}trimethylammonium iodide

Step A:

### Methyl 4-hydroxy-2-methyl-1,1-dioxo-3,4-dihydro-2H-1,2-benzothiazine-3carboxylate

10 mmol of sodium borohydride are added to 10 mmol of methyl 4-hydroxy-2-methyl-1,1-dioxo-2H-1,2-benzothiazine-3-carboxylate (the preparation of which is described in J. Med. Chem. 1999, 42, 5235-40) in methanol, and then the reaction mixture is stirred for 2 hours. After removal of the solvent by evaporation, the resulting residue is hydrolysed and then extracted with ethyl acetate. The combined organic phases are washed, dried, filtered and then concentrated. The resulting residue is purified by chromatography over silica (eluant:dichloromethane) to yield the expected product.

Step B:

### {3-[(4-Hydroxy-2-methyl-1,1-dioxo-3,4-dihydro-2H-1,2-benzothiazin-3-yl)carbonylamino]propyl}trimethylammonium iodide

The expected product is obtained according to the process described in Steps B and C of Example 5, starting from the compound described in the preceding Step.

### EXAMPLE 8

### {3-[(4-Acetoxy-2-methyl-1,1-dioxo-3,4-dihydro-2H-1,2-benzothiazin-3-yl)carbonylamino]propyl}trimethylammonium iodide

Step A:

### Methyl 4-acetoxy-2-methyl-1,1-dioxo-3,4-dihydro-2H-1,2-benzothiazine-3carboxylate

The expected product is obtained according to the process described in Step A of Example 6, starting from the compound described in Step A of Example 7.

Step B:

### {3-[(4-Acetoxy-2-methyl-1,1-dioxo-3,4-dihydro-2H-1,2-benzothiazin-3-yl)carbonylamino]propyl}trimethylammonium iodide

The expected product is obtained according to the process described in Steps B and C of Example 5, starting from the compound described in the preceding Step.

### EXAMPLE 9

### {3-[(2-Methyl-1,1-dioxo-2H-1,2-benzothiazin-3-yl)carbonylamino]propyl}trimethylammonium iodide

Step A:

### Methyl 2-methyl-1,1-dioxo-2H-1,2-benzothiazine-3-carboxylate

10 mmol of 1,8-diazabicyclo[5.4.0]undec-7-ene are added to 10 mmol of the compound described in Step A of Example 8 dissolved in tetrahydrofuran, and then the reaction mixture is stirred at room temperature for 4 hours. After hydrolysis and extraction with ethyl acetate, the combined organic phases are washed, dried, filtered and then concentrated. The resulting residue is purified by chromatography over silica (eluant: dichloromethane) to yield the expected product.

Step B:

### {3-[(2-Methyl-1,1-dioxo-2H-1,2-benzothiazin-3-yl)carbonylamino]propyl}trimethylammonium iodide

The expected product is obtained according to the process described in Steps B and C of Example 5, starting from the compound described in the preceding Step.

### EXAMPLE 10

### {3-[(2-Methyl-1,1-dioxo-3,4-dihydro-2H-1,2-benzothiazin-3yl)carbonylamino]propyl}trimethylammonium iodide

Step A:

### Methyl 2-methyl-1,1-dioxo-3,4-dihydro-2H-1,2-benzothiazine-3-carboxylate

A solution of the compound described in Step A of Example 9 (10 mmol) in methanol is placed under hydrogen overnight in the presence of 10% Pd/C. After removal of the catalyst by filtration, the solvent is removed by evaporation to yield the expected product.

Step B:

### {3-[(2-Methyl-1,1-dioxo-3,4-dihydro-2H-1,2-benzothiazin-3-yl)carbonylamino]propyl}trimethylammonium iodide

The expected product is obtained according to the process described in Steps B and C of Example 5, starting from the compound described in the preceding Step.

### EXAMPLE 11

### {3-[(4-(para-Toluenesulphonyloxy)-2-methyl-1,1-dioxo-2H-1,2benzothiazin-3-yl)carbonylamino]propyl}trimethylammonium iodide

Step A:

### Methyl 2-methyl-4-(para-toluenesulphonyloxy)-1,1-dioxo-2H-1,2benzothiazine-3-carboxylate

The expected product is obtained according to the process described in Step A of Example 6, replacing acetyl chloride by para-toluenesulphonyl chloride.

Step B:

### {3-[(4-(para-Toluenesulphonyloxy)-2-methyl-1,1-dioxo-2H-1,2-benzothiazin-3yl)carbonylamino]propyl}trimethylammonium iodide

The expected product is obtained according to the process described in Steps B and C of Example 5, starting from the compound described in the preceding Step A.

### EXAMPLE 12

### {3-[(4-(Methanesulphonyloxy-2-methyl-1,1-dioxo-2H-1,2-benzothiazin-3-yl) carbonylamino]propyl}trimethylammonium iodide

Step A:

### Methyl 2-methyl4-(methanesulphonyloxy)-1,1-dioxo-2H-1,2-benzothiazine-3carboxylate

The expected product is obtained according to the process described in Step A of Example 6, replacing acetyl chloride by methanesulphonyl chloride.

Step B:

### {3-[(4-(Methanesulphonyloxy)-2-methyl-1,1-dioxo-2H-1,2-benzothiazin-3yl)carbonylamino]propyl}trimethylammonium iodide

The expected product is obtained according to the process described in Steps B and C of Example 5, starting from the compound described in the preceding Step A.

### EXAMPLE 13

### {3-[(4-Benzyloxy-2-methyl-1,1-dioxo-2H-1,2-benzothiazin-3yl)carbonylamino]propyl}trimethylammonium iodide

Step A:

### Methyl 4-benzyloxy-2-methyl-1,1-dioxo-2H-1,2-benzothiazine-3-carboxylate

The expected product is obtained according to the process described in Step A of Example 6, replacing acetyl chloride by benzyl chloride.

Step B:

### {3-[(4-Benzyloxy-2-methyl-1,1-dioxo-2H1,2-benzothiazin-3-yl)carbonylamino]propyl}trimethylammonium iodide

The expected product is obtained according to the process described in Steps B and C of Example 5, starting from the compound described in the preceding Step A.

### EXAMPLE 14

### {3-[(4-Hydroxy-2-methyl-1,1-dioxo-2H-1,2-benzothiazin-3yl)carbonylamino]propyl}diethylmethylammonium bromide

The expected product is obtained according to the process described in Step B of Example 1, starting from the compound obtained in Step A of Example 1 and bromomethane.

### EXAMPLE 15

### {3-[(4-Hydroxy-2-methyl-1,1-dioxo-2H-1,2-benzothiazin-3yl)carbonylamino]propyl}diethylmethylammonium chloride

The expected product is obtained according to the process described in Step B of Example 1, starting from the compound obtained in Step A of Example 1 and chloromethane.

### PHARMACOLOGICAL STUDY OF THE COMPOUNDS OF THE INVENTION

### EXAMPLE 16

### Pharmacokinetic Study: Tissue Distribution Study

This study was carried out with molecules labelled with ¹⁴C. The tissue distribution study was carried out by direct measurement of the radioactivity across whole-body sections in accordance with the following method:male rats of the Sprague-Dawley strain were administered intravenously or orally with a dose of the labelled molecule. Then, after periods ranging from 5 minutes to 24 hours, the animals were sacrificed by ether inhalation and frozen in liquid nitrogen.

Sections were then prepared using a cryomicrotome and, after dessiccation, the distribution of the radioactivity was measured using an image analyser.

The results obtained with the compounds of the invention demonstrate that the compounds exhibit an increased tropism for cartilaginous tissues.

### EXAMPLE 17

### Expression of Aggrecan in Articular Chondrocytes Treated with IL-1

The expression of aggrecan was analysed by Northern blot. Total RNA of cultures of calf articular chondrocytes (CAC) cultured in DMEM+10% FCS and treated with different concentrations of the test compounds, in the presence of IL-1 (10 ng/ml), were extracted, 10 μg of the total RNA were fractionated by electrophoresis on a 1% agarose gel in the presence of an MOPS-formaldehyde buffer, transferred to a nylon membrane and hybridised with a specific aggrecan cDNA probe.

By way of example, the compound of Example 1, at 10⁻⁶ and 10⁻M, stimulates the expression of aggrecan by 150% and 200%, respectively, compared with cultures treated with 10 ng/ml of IL-1.

By way of comparison, “propoxicam N⁺” described in the publication J. Med. Chem. 1999, 42, 5235-5240, at 10⁻⁶ and 10⁻⁸M, stimulates the expression of aggrecan by only 28% and 30%, respectively, under the same conditions.

These results demonstrate that the compounds of the invention have very valuable cartilage-protecting properties.

### EXAMPLE 18

### Pharmaceutical Composition

Formulation for the preparation of 1000 tablets each containing a dose of 10 mg of active ingredient

| | |
|---|---|
| Compound of Example 1 | 10 g |
| Hydroxypropylcellulose | 2 g |
| Wheat starch | 10 g |
| Lactose | 100 g |
| Magnesium stearate | 3 g |
| Talc | 3 g |

1. Compound of formula (I): 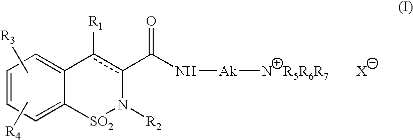
wherein: [symbol]
represents a single or double bond,
R₁ represents a hydrogen atom or a hydroxy group, a linear or branched (C₁-C₆)alkoxy group, a linear or branched (C₁-C₆)acyloxy group, a linear or branched (C₁-C₆)alkylsulphonyloxy group, an arylsulphonyloxy group or an aryl-(C₁-C₆)alkoxy group in which the alkoxy moiety is linear or branched,
R₂ represents a hydrogen atom or a linear or branched (C₁-C₆)alkyl group,
R₃ and R₄, which may be identical or different, each represent a hydrogen atom, a halogen atom or a linear or branched (C₁-C₆)alkyl group, a hydroxy group or a linear or branched (C₁-C₆)alkoxy group,
Ak represents a linear or branched (C₁-C₆)alkylene chain,
R₅, R₆ and R₇, which may be identical or different, each represent a linear or branched (C₁-C₆)alkyl group,
or R₅, R₆ and R₇, taken together with the nitrogen atom carrying them, form a saturated or unsaturated nitrogen-containing heterocycle,
X represents a halogen atom, and its optical isomers when they exist, excluding compounds wherein, simultaneously, [symbol] represents a double bond, R₁ represents a hydroxy group, R₂, R₅ and R₆ each represent a methyl group, R₃ and R₄ each represent a hydrogen atom, and Ak represents a —(CH)₃— group,
it being understood that “saturated or unsaturated nitrogen-containing heterocycle” is to be understood as meaning a saturated or unsaturated, aromatic or non-aromatic, monocyclic group having from 5 to 7 ring members, containing one, two or three hetero atoms, one of those hetero atoms being a nitrogen atom, and the additional hetero atom(s) that is/are optionally present being selected from the atoms oxygen, nitrogen and sulphur, it being understood that the nitrogen-containing heterocycle can optionally be substituted by one or more identical or different linear or branched (C₁-C₆)alkyl groups. The preferred nitrogen-containing heterocycles are the groups pyridyl and piperidyl that is N-substituted by a linear or branched (C₁-C₆)alkyl group.

2. Compound of formula (I) according to claim 1, wherein R₂ represents a linear or branched (C₁-C₆)alkyl group.

3. Compound of formula (I) according to either claim 1 or claim 2, wherein R₅, R₆ and R₇, which may be identical or different, each represent a linear or branched (C₁-C₆)alkyl group.

4. Compound of formula (1) according to claim 3, wherein R₆ and R₇, which may be identical or different, each represent a linear or branched (C₂-C₆)alkyl group.

5. Compound of formula (1) according to either claim 1 or claim 2, wherein R₅, R₆ and R₇, taken together with the nitrogen atom carrying them, form a saturated or unsaturated nitrogen-containing heterocycle.

6. Compound of formula (I) according to claim 5, wherein R₅, R₆ and R₇, taken together with the nitrogen atom carrying them, form a pyridyl group.

7. Compound of formula (I) according to any one of claims 1 to 6, wherein X represents an iodine atom.

8. Compound of formula (I) according to claim 1, which is {3-[(4-hydroxy-2-methyl-1,1dioxo-2H-1,2-benzothiazin-3-yl)-carbonylamino]-propyl}-diethylmethylammonium iodide.

9. Process for the preparation of compounds of formula (I) according to claim 1, characterised in that a compound of formula (II): 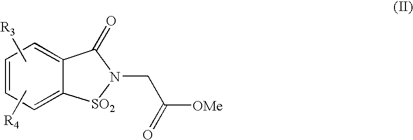
wherein R₃ and R₄ are as defined for formula (I), is reacted in the presence of a base, to yield a compound of formula (III): 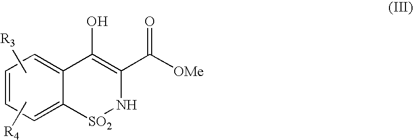
wherein R₃ and R₄ are as defined hereinbefore, which is reacted, if desired, with a compound of formula (IV):
R′₂—Y (IV)
wherein R′₂ represents a linear or branched (C₁-C₆)alkyl group, and Y₁ represents a leaving group conventional in organic chemistry, to yield a compound of formula (V): 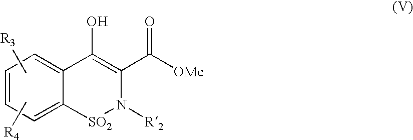
wherein R′₂, R₃ and R₄ are as defined hereinbefore,
which compounds of formula (III) or (V) are reacted, if desired,
with a compound of formula (VI):
R′₁—Y₂ (VI)
wherein R′₁ represents a linear or branched (C₁-C₆)alkyl group, a linear or branched (C₁-C₆)acyl group, a linear or branched (C₁-C₆)alkylsulphonyl group, an arylsulphonyl group or an aryl-(C₁-C₆)alkyl group in which the alkyl moiety is linear or branched, and Y2 represents a leaving group customary in organic chemistry, to yield a compound of formula (VII): 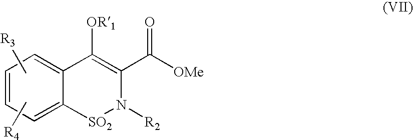
wherein R′₁, R₂, R₃ and R₄ are as defined hereinbefore,
or with an appropriate reducing agent, to yield a compound of formula (VIII): 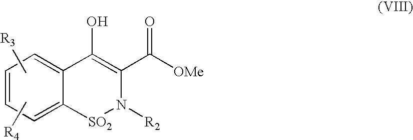
wherein R₂, R₃ and R₄ are as defined hereinbefore, which is optionally converted:
by elimination, to a compound of formula (IX), under conventional conditions of organic chemistry: 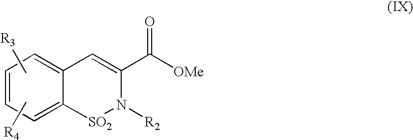
wherein R₂, R₃ and R₄ are as defined hereinbefore, which is reduced, if desired, to a compound of formula (X): 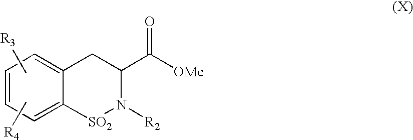
wherein R₂, R₃ and R₄ are as defined hereinbefore,
or by reaction with a compound of formula (VI), to yield a compound of formula (XI): 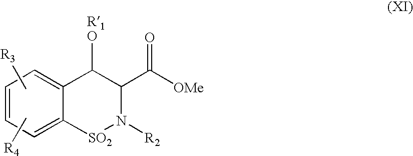
wherein R′₁ , R₂, R₃ and R₄ are as defined hereinbefore,
which compounds of formulae (III), (V), (VII), (VIII), (IX), (X) and (XI) constitute the totality of the compounds of formula (XII): 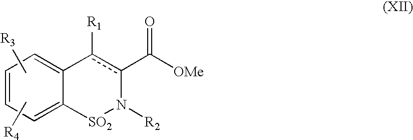
wherein [symbol] , R₁, R₂, R₃ and R₄ are as defined for formula (I), which are converted to a compound of formula (XIII): 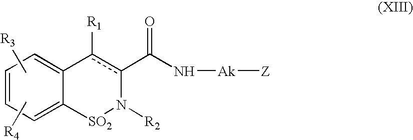
wherein R₁, R₂, R₃ and R₄ are as defined hereinbefore, Ak is as defined for formula (I), and Z represents either a group X as defined for formula (I) or a group NR′₅R′₆ wherein R′₅ and R′₆, which may be identical or different, each represent a linear or branched (C₁-C₆)alkyl group or together form a saturated or unsaturated, non-aromatic nitrogen-containing heterocycle,
which is reacted:
when Z represents a group NR′₅R′₆ as defined hereinbefore, with a compound of formula (XIV):
R′₇—X (XIV)
wherein R′₇ represents a linear or branched (C₁-C₆)alkyl group, and X is as defined for formula (I),
to yield a compound of formula (Ia), a particular case of the compounds of formula (I): 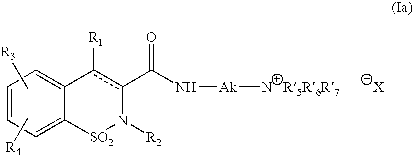
wherein R₁, R₂, R₃, R₄, Ak, R′₅, R′₆, R′₇ and X are as defined hereinbefore,
or, when Z represents a group X as defined hereinbefore, with a compound of formula (XV):
NR″₅R″₆R″₇ (XV)
wherein R″₅, R″₆ and R″₇ form together with the nitrogen atom carrying them an aromatic nitrogen-containing heterocycle,
to yield a compound of formula (Ib), a particular case of the compounds of formula (I): 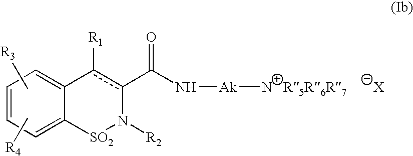
wherein R₁, R₂, R₃, R₄, Ak, R″₅, R″₆, R″₇ and X are as defined hereinbefore,
which compounds of formulae (Ia) and (Ib) constitute the totality of the compounds of formula (I), which are purified, if necessary, in accordance with a conventional purification technique, and which are separated, where appropriate, into their optical isomers in accordance with a conventional separation technique.

10. Pharmaceutical composition comprising as active ingredient a compound according to any one of claims 1 to 8, in combination with one or more inert, non-toxic, pharmaceutically acceptable carriers.

11. Pharmaceutical composition according to claim 10 for use as a medicament in the treatment of arthrosis or arthritis.
